(19) United States
(12) Patent Application Publication
(10) Pub. No.: US 2025/0087375 A1
(43) Pub. Date: Mar. 13, 2025

(54) INFORMATION INTERACTION

(71) Applicant: Beijing Zitiao Network Technology Co., Ltd., Beijing (CN)

(72) Inventors: Wei Tu, Beijing (CN); Yihang Tang, Beijing (CN)

(21) Appl. No.: 18/882,648

(22) Filed: Sep. 11, 2024

(30) Foreign Application Priority Data
Sep. 11, 2023 (CN) 202311167462.8

(51) Int. Cl. G16H 80/00; G16H 10/20

(52) U.S. Cl. CPC G16H 80/00; G16H 10/20

(57) ABSTRACT

The embodiment of the present disclosure provides a solution for information interaction. The method includes: in response to a consultation initiating operation, providing an interaction interface between a user and a digital assistant for health consultation from the user; receiving a target file input by the user, the target file being in a non-text message format; determining, based on an interaction context between the user and he digital assistant, whether the target file meets a health consultation requirement; and in response to determining that the target file meets the health consultation requirement, determining an analysis result of the digital assistant for the target file based on information included in the target file.

### CROSS-REFERENCE TO RELATED APPLICATION(S)

The present disclosure claims priority to Chinese Patent Application No. 202311167462.8 filed on Sep. 11, 2023, and entitled “METHOD AND APPARATUS FOR INFORMATION INTERACTION, DEVICE AND STORAGE MEDIUM”.

### FIELD

Example embodiments of the present disclosure generally relate to the field of computers, and in particular, to a method for information interaction, a device, and a computer-readable storage medium.

### BACKGROUND

With the development of Internet technologies, it is also expected that people will be able to enjoy remote and online services in terms of health consultation. Online medical care improves the supply of high-quality medical resources at high efficiency and low cost, helps the medical institution to expand the radiation range of medical service, and enables the consultation recipients to obtain authoritative and professional medical guidance at any time. Currently, some websites, terminal applications, etc. attempt to provide online health consultation services. However, in online health consultation, there is an improvement in interactivity and expertise.

### SUMMARY

In a first aspect of the present disclosure, a method for information interaction is provided. The method includes: in response to a consultation initiating operation, providing an interaction interface between a user and a digital assistant for health consultation from the user; receiving a target file input by the user, the target file being in a non-text message format; determining, based on an interaction context between the user and the digital assistant, whether the target file meets a health consultation requirement; and in response to determining that the target file meets the health consultation requirement, determining an analysis result of the digital assistant for the target file based on information included in the target file.

In a second aspect of the present disclosure, an apparatus for information interaction is provided. The apparatus includes an interface providing module configured to provide, in response to a consultation initiating operation, an interaction interface between a user and a digital assistant for health consultation from the user; a file receiving module configured to receive a target file input by the user, the target file being in a non-text message format; a request determining module configured to determine, based on an interaction context between the user and the digital assistant, whether the target file meets a health consultation requirement; and an analysis determining module configured to determine, in response to determining that the target file meets the health consultation requirement, an analysis result of the digital assistant for the target file based on information included in the target file.

In a third aspect of the present disclosure, an electronic device is provided. The device includes at least one processing unit; and at least one memory coupled to the at least one processing unit and storing instructions for execution by the at least one processing unit. The instructions, when executed by the at least one processing unit, cause the device to implement the method of the first aspect.

In a fourth aspect of the present disclosure, a computer-readable storage medium is provided. The medium stores a computer program thereon, and when executed by the processor, the computer program implements the method in the first aspect.

It should be understood that the content described in this section is not intended to limit the key features or important features of the embodiments of the present disclosure, nor is it intended to limit the scope of the present disclosure. Other features of the present disclosure will become readily understood from the following description.

### BRIEF DESCRIPTION OF DRAWINGS

The above and other features, advantages, and aspects of various embodiments of the present disclosure will become more apparent from the following detailed description taken in conjunction with the accompanying drawings. In the drawings, the same or similar reference numbers refer to the same or similar elements, wherein:

FIG. 1 illustrates a schematic diagram of an example environment in which embodiments of the present disclosure can be implemented;

FIG. 2A to FIG. 2D illustrates schematic diagrams of examples of interaction interfaces according to some embodiments of the present disclosure;

FIG. 3A to FIG. 3D illustrates schematic diagrams of examples of interaction interfaces according to some other embodiments of the present disclosure;

FIG. 4A to FIG. 4D illustrates schematic diagrams of examples of interaction interfaces according to still other embodiments of the present disclosure;

FIG. 5 illustrates a flowchart of a process for information interaction according to some embodiments of the present disclosure;

FIG. 6 illustrates a block diagram of an apparatus for information interaction according to some embodiments of the present disclosure; and

FIG. 7 illustrates a block diagram of an electronic device in which one or more embodiments of the present disclosure may be implemented.

### DETAILED DESCRIPTION

Embodiments of the present disclosure will be described in more detail below with reference to the accompanying drawings. While certain embodiments of the present disclosure are shown in the accompanying drawings, it should be understood that the present disclosure may be implemented in various forms, and should not be construed as limited to the embodiments set forth herein, but rather, these embodiments are provided for a more thorough and complete understanding of the present disclosure. It should be understood that the drawings and embodiments of the present disclosure are for exemplary purposes only and are not intended to limit the scope of the present disclosure.

In the description of the embodiments of the present disclosure, the terms “including” and the like should be understood to include “including but not limited to”. The term “based on” should be understood as “based at least in part on”. The terms “one embodiment” or “the embodiment” should be understood as “at least one embodiment”. The term “some embodiments” should be understood as “at least some embodiments”. Other explicit and implicit definition may also be included below.

Herein, unless explicitly stated, “responding to A” performs one step and does not imply that this step is performed immediately after “A”, but may include one or more intermediate steps.

It may be understood that the data involved in the technical solution (including but not limited to the data itself, the obtaining, using, storing or deleting of the data) should follow the requirements of the corresponding laws and regulations and related regulations.

It can be understood that before using the technical solutions disclosed in the embodiments of the present disclosure, relevant user should be informed of the types, use ranges, usage scenarios, and the like of the information related to the present disclosure in an appropriate manner according to relevant laws and regulations, and the authorization of the related user may be obtained, wherein the relevant users may include any type of subject of rights, such as individuals, businesses, and groups.

For example, in response to receiving an active request from a user, prompt information is sent to the user to explicitly prompt the user that an operation requested by the user will require obtaining and use of personal information of the user. Thus, the user can autonomously select, according to the prompt information, whether to provide personal information to software or hardware such as an electronic device, an application program, a server, or a storage medium that executes the operations of the technical solutions of the present disclosure.

As an optional but non-limiting implementation, in response to receiving an active request from the user, prompt information is sent to the user, for example, in the form of a pop-up window, and the pop-up window may present the prompt information in the form of text. In addition, the pop-up window may also carry a selection control for the user to select whether he/she “agrees” or “disagrees” to provide personal information to the electronic device.

It can be understood that the above notification and user authorization process are only illustrative which do not limit the implementation of this disclosure. Other methods that meet relevant laws and regulations can also be applied to the implementation of this disclosure.

As used herein, the term “model” may learn association between corresponding inputs and outputs from training data, so that after the training is complete, a corresponding output may be generated for a given input. The generation of the model may be based on a machine learning technology. Depth learning is a machine learning algorithm that processes inputs and provides corresponding outputs by using a multi-tiered processing unit. A neural network model is one example of a model based on deep learning. Herein, “model” may also be referred to as “machine learning model”, “learning model”, “machine learning network”, or “learning network”, which may be used interchangeably herein.

In an online health consultation process, a user may have some data expected to be directly uploaded for consultation, such as examination reports, medical records and the like. However, most model-based digital assistants do not support processing information in a non-text message format. In addition, in practical applications, the user may upload non-medical health related pictures/files, or the photos shot by the user may be not standardized, which may cause the model not being able to accurately identify the photos and provide useful feedback.

According to an embodiment of the present disclosure, an improved solution for information interaction is provided. In this solution, during an interaction process between the user and the digital assistant for health consultation, if the user inputs a file in the non-text message format, it is determined whether the file meets the health consultation requirement based on the interaction context between the user and the digital assistant. When it is determined that the file meets the health consultation requirement, an analysis result of the digital assistant for the file is determined based on the information extracted from the file. In an embodiment of the present disclosure, for a file in a non-text message format, the context is used to determine whether the file meets the requirement, and then the file meeting the requirement is analyzed. This may effectively avoid providing an analysis result of the health file in a case where the file does not meet the requirement, and thus causing inaccurate feedback results, misleading, and affecting reliability of health consultation and user friendliness.

Some example embodiments of the present disclosure will be described in detail below with reference to the accompanying drawings.

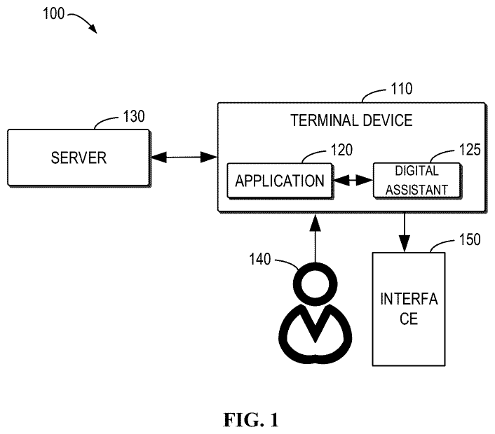

FIG. 1 illustrates a schematic diagram of an example environment 100 in which embodiments of the present disclosure can be implemented. In this example environment 100, an application 120 is installed in the terminal device 110. The user 140 may interact with the application 120 via the terminal device 110 and/or an attachment device of the terminal device 110.

In some embodiments, the application 120 may be downloaded, installed on the terminal device 110. In some embodiments, the application 120 may also be accessed in other manners, such as may be accessed via web pages. In the environment 100 of FIG. 1, if the application 120 is initiated, the terminal device 110 may present the interface 150 of the application 120. The interface 150 may include various user interfaces that may be provided by the application 120. The interface 150 is also sometimes referred to as a client interface.

In embodiments of the present disclosure, the application 120 includes any suitable application capable of providing a health consultation service. In some embodiments, the application 120 may implement a dialog interaction with the user 140 based on the digital assistant 125 to provide a health consultation service to the user 140. In some embodiments, the application 120 may provide other services in addition to interaction with the digital assistant 125, which is not limited in the embodiments of the present disclosure.

In some embodiments, the terminal device 110 may communicate with server 130 to enable provision of services to the application 120 and/or the digital assistant 125. The terminal device 110 may be any type of mobile terminal, fixed terminal, or portable terminal, including a mobile phone, a desktop computer, a laptop computer, a notebook computer, a netbook computer, a tablet computer, a media computer, a multimedia tablet, a personal communication system (PCS) device, a personal navigation device, a personal digital assistant (PDA), an audio/video player, a digital camera/camcorder, a television receiver, a radio broadcast receiver, an electronic book device, a gaming device, or any combination of the foregoing, including accessories and peripherals of these devices, or any combination thereof. In some embodiments, the terminal device 110 can also support any type of interface for a user (such as a “wearable” circuit, etc.). The applications 130 may be various types of computing systems/servers capable of providing computing power, including, but not limited to, mainframes, edge computing nodes, computing devices in a cloud environment, and so forth.

It shall be understood that the structures and functions of the various elements in the environment 100 are described for exemplary purposes only, which do not imply any limitation to the scope of the present disclosure.

In an embodiment of the present disclosure, the digital assistant 125 is configured to interact with a user. An interaction interface (also referred to as an interaction window or a dialog window) between the user and the digital assistant 125 is presented in the client interface. The digital assistant 125, as an intelligent assistant, has an intelligent dialogue and information processing capability. In the interaction interface, the user enters an interaction message, and the digital assistant 125 provides a reply message in response to user input. In the application 120, the digital assistant 125 may be initiated or awakened in an appropriate manner (e.g., a shortcut key, button, or voice) to present an interaction interface with the user. The interaction interface may include an interface element for information interaction, such as an input box, a message list, a message bubble, and the like.

In embodiments of the present disclosure, the digital assistant 125 is configured to provide services related to health consultation, and the digital assistant 125 may sometimes be referred to as a “digital doctor”. A user may send a health consultation request to the digital assistant 125 by inputting simple text or voice, and may obtain a real-time reply from the digital assistant 125 for medical knowledge science Q&A, health consultation, or general health management. In this way, the digital assistant 125 can be used to improve the supply of high-quality medical resources in high efficiency and low cost, to help the medical institution to expand the radiation range of the medical service, and enable the user to obtain authoritative and professional guidance at any time.

In some embodiments, the digital assistant 125 may determine a reply to the user's question by means of a model. In some embodiments, the model may be trained based on a general corpus and a specialized corpus in the medical field, enabling reachable, trustworthy, understandable medical knowledge publicity and health consultation guidance. The specialized corpus in the medical field may include, for example, domestic and foreign authoritative clinical guidelines, literatures, textbooks, clinical doctor-patient communication Q&A data, and the like. In some embodiments, training of the medical safety related mechanism can be strengthened during model training, so that the model can have a capability of providing credible health consultation suggestions, and a comprehensive safety test is achieved. In some embodiments, when evaluating the performance of the digital assistant 125, the capability of the digital assistant 125 in terms of communication expression ability and humanistic care and friendliness may also be evaluated, so that the digital assistant 125 can realize aspects such as empathizing the user, understanding the user's needs, and communicating in detail and patience, which is conducive to alleviating the problems of short communication time and inadequate communication between doctors and patients.

The model may be deployed on the terminal device 110 or may be accessed by the terminal device 110. Alternatively, the terminal device 110 may communicate with the model, so as to provide an application management service for the user 140 by using the model. For example, the terminal device 110 may send a prompt word to the model based on the interaction information between the user 140 and the digital assistant 125, and obtain a processing result of the model. Alternatively or additionally, the digital assistant 125 itself may also be obtained based on a model. The digital assistant 125 may process a conversation message input by the user 140 to obtain a requirement from the user 140, for example, a problem expected to be consulted by the user 140, and the like. The model may be configured locally at the terminal device 110 or independently of terminal device 110 as needed, and the present disclosure is not intended to be limited thereto.

In an embodiment of the present disclosure, in response to a consultation initiating operation, an interaction interface between the user 140 and the digital assistant 125 is provided for health consultation from the user 140. The consultation initiating operation may be an operation initiated by the user 140 in any suitable manner. The health consultation may be a health consultation with the user 140 as a consulting object, or the consulting object may be specified by the user 140. In an interaction interface between the user 140 and the digital assistant 125, the user may be supported to input various types of interaction messages through text or voice. The voice input may be converted to a text message. The digital assistant 125 determines a corresponding reply based on the text input by the user.

In some cases, the user 140 may input a file in a non-text message format for consultation. In an embodiment of the present disclosure, the user is supported to input a message in multimodal data format for interaction, including a text message format and a non-text message format. When the user 140 inputs a file in a non-text message format, the digital assistant 125 can also properly determine the reply and provide an analysis result for the text. In this way, the user can be supported to upload or shoot an existing medical report or other medical related information for medical consultation, thereby improving interaction efficiency.

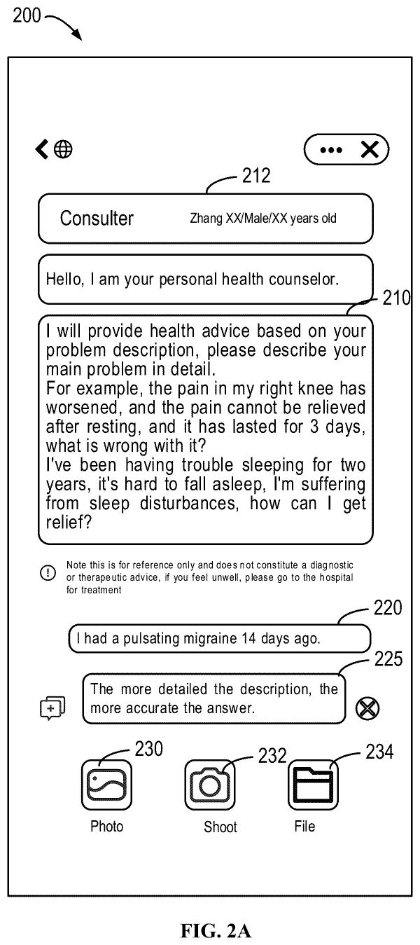

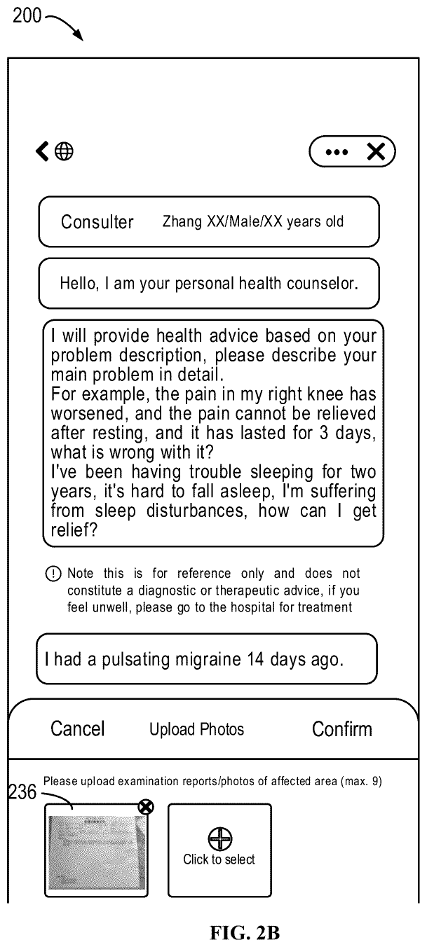

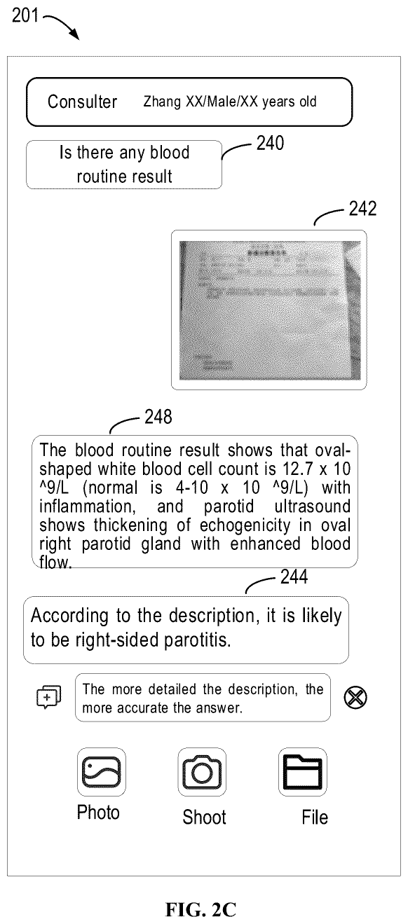

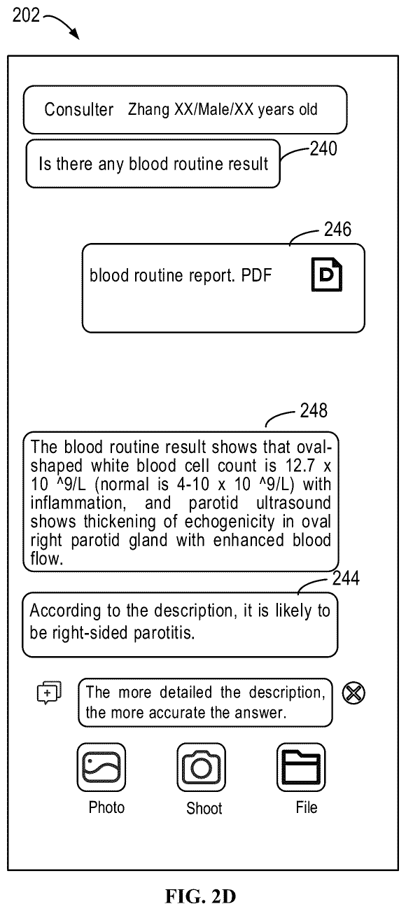

Hereinafter, for a better understanding of the embodiments of the present disclosure, descriptions will be provided with reference to the examples of the interaction interfaces shown in FIGS. 2A-2D, 3A-3B, and 4A-4D. The interaction interfaces of these drawings may be provided by the terminal device 110 to the user 140. It shall be understood that the interaction interfaces in these drawings are merely examples, and various interface designs may actually exist. Individual graphical elements in the interface may have different arrangements and different visual representations, one or more of which may be omitted or replaced, and one or more other elements may also be present. Embodiments of the present disclosure are not limited in this respect.

The interaction interface 200 of FIG. 2A may be presented to the user 140 in response to a consultation initiating operation. In the interaction interface 200, the digital assistant 210 may provide a message 210 to guide the user 140 for consultation. The user 140 may input the text message 220 through the input box 225 to describe a question to be consulted in a text manner. In some embodiments, identification information of a current consultation object may also be shown in a predetermined area 212 of the interaction interface, and the consultation object may be the current user 140 or another user having an association relationship with the user 140, which is not limited herein.

In an embodiment of the present disclosure, in addition to supporting a user inputting a message in a text or voice manner, a user is further supported to input a file in a non-text message format. Files in the non-textual message format may include various types of electronic files, including medical reports in electronic format or electronically scanned or captured version of paper medical reports.

As shown in FIG. 2A, the user 140 may also upload a locally existing image file or video file through the “Photo” control 230, shoot an image file or video file in real time through the “Shooting” control 232, or upload the existing other files stored locally through the “File” control 234, such as a document file, a txt file, a PDF file, an Excel file, or files in any other suitable format. As shown in FIG. 2A, if the user selects the “Photo” control 230, one or more local images or video files 236 may be selected to be uploaded for inputting into the interaction interface 200.

After receiving a target file input by the user 140, if the target file is in a non-text message format, the terminal device 110 may determine, based on the interaction context between the user 140 and the digital assistant 125, whether the target file meets the health consultation requirement. Since the reply of the digital assistant 125 is determined by operating a model, if the messages or files input by all the users are directly provided to the model for processing, when a file input by a user does not meet the requirement, a situation that the reply does not meet the expectation and thus cannot provide useful feedback may occur. Unlike manually inputting a text message or inputting voice by a user, when a file is shot or uploaded, there may be situations such as wrong selection, inaccurate shooting, and the like. For such errors, the digital assistant 125 is expected to be able to provide more intelligent feedback to guide the user.

In some embodiments, when determining whether the target file meets the health consultation requirement, it may be determined whether the target file includes medical related information, for example, it may be determined whether a file name of the target file, metadata of the target file, or information parsed from the target file corresponds to the medical related information. In general context of health consultation, it is desirable that the file input by the user 140 should be information associated with the health consultation, so as to avoid the interaction with the digital assistant 125 being directed to an unrelated direction.

In some embodiments, alternatively or additionally, when determining whether the target file meets the health consultation requirement, it may be determined whether an information type included in the target file matches an information type requested from the user 140 in the interaction context. For example, in interaction between the user 140 and the digital assistant 125, the user 140 may actively be requested to provide some type of medical related information, or the digital assistant 125 may request the user 140 to provide some type of medical related information.

In some embodiments, alternatively or additionally, when it is determined whether the target file meets the health consultation requirement, it may be determined whether an appearance layout of the target file meets a predetermined layout requirement. This is particularly suitable for the case where the target file is generated in a shooting or scanning manner, and due to the angle for shooting or scanning, the file may not include complete information of the medical report, the file may have distortion and deformation, or the display of content is unclear due to dark shadow; and thus the target file may not meet the requirement on the appearance layout of the target file. In order to ensure that subsequent analysis is accurate, it is desirable for the user to modify or provide a more accurate target file.

In some embodiments, when it is determined that the target file includes medical related information, the included information type matches the information type requested from the user 140 in the interaction context, and/or the appearance layout of the target file meets the predetermined layout requirement, it is determined that the target file meets the health consultation requirement. In this case, the analysis result of the digital assistant 125 for the target file may be determined based on the information included in the target file.

As shown in the interaction interface 201 of FIG. 2C, the digital assistant 125 provides a message 240 in the interaction interface to request the user to provide an examination result of “blood routine”. The user 140 uploads an image file 242. By parsing the image file 242, it can be determined that the image file 242 contains a blood routine result, so the file meets the health consultation requirement. Next, the digital assistant 125 provides an analysis result 244 based on the information included in the file and other information provided by the user in the interaction context. Similarly, in the interaction interface 202 of FIG. 2D, the user 140 uploads the PDF file 246. By parsing the PDF file 246, it may be determined that the image file 242 contains a blood routine result, so the file meets to the health consultation requirement, and the analysis result 244 may be similarly provided.

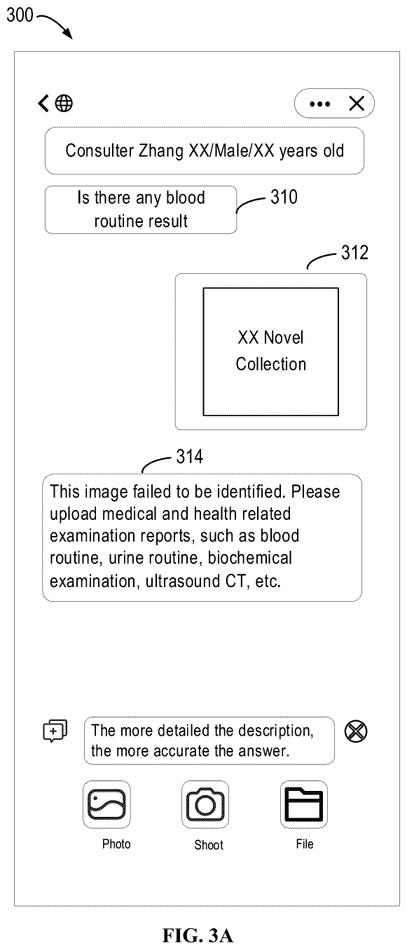

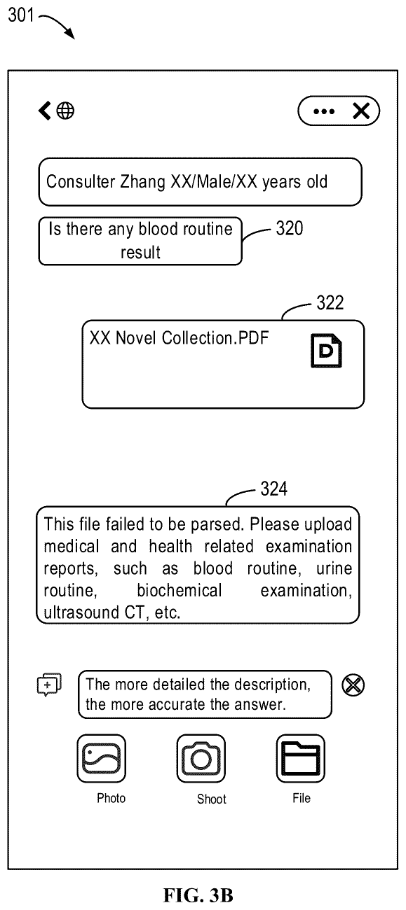

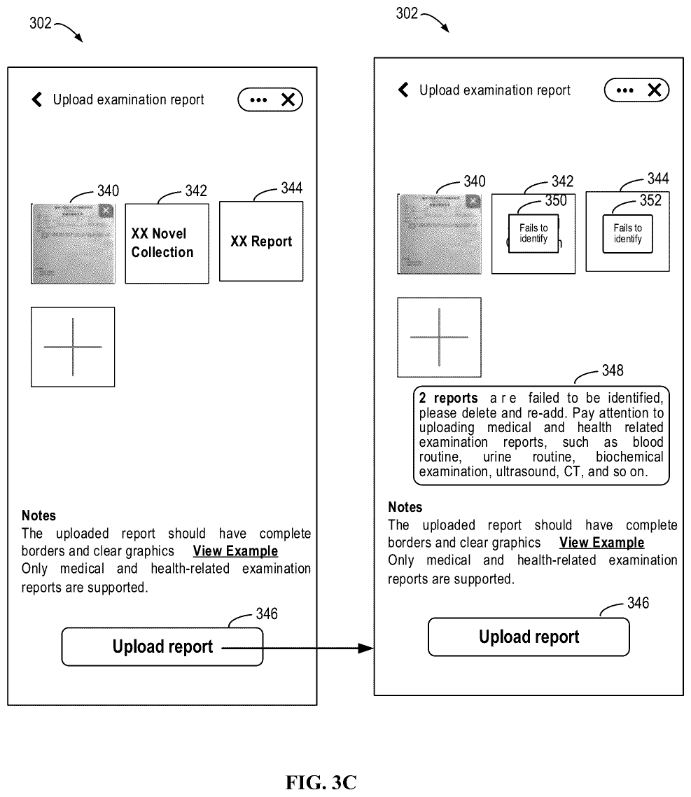

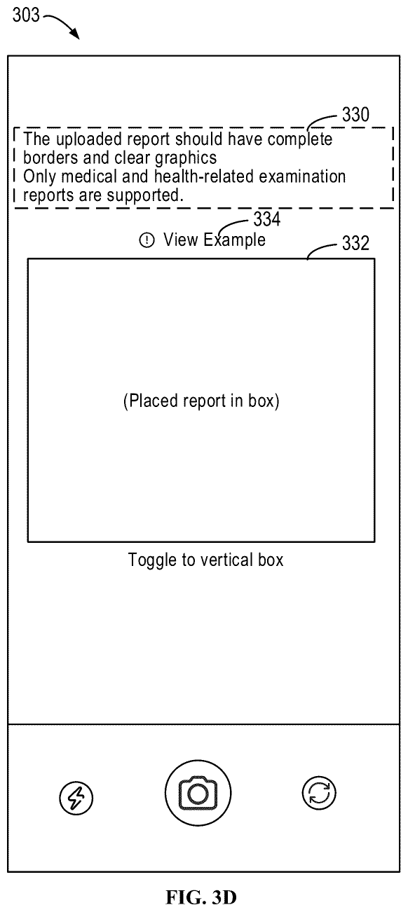

In some embodiments, in response to determining that the target file does not meet the health consultation requirement, the terminal device 110 may provide prompt information to prompt the user 140 to input a correct file. In some embodiments, the target file is provided as an interaction message in the interaction interface between the user 140 and the digital assistant 125, and the prompt information may also be provided as an interaction message in the interaction interface. As shown in FIG. 3A, in the interaction interface 300, the digital assistant 125 provides a message 310 in the interaction interface to request the user to provide a result of “ultrasound examination”. The user 140 uploads an image file 312. By parsing the image file 312, for example, by extracting the text contained in the image file 312 or identifying the layout of the image file 312, it can be determined that the image file 312 does not relate to “ultrasonic examination”, so it can be determined that the file does not meet the health consultation requirement. The digital assistant 125 may provide prompt information 314 to indicate that the current file does not meet the requirement, and prompt the user 140 to input a correct file. Similarly, in an interaction interface 301 shown in FIG. 3B, the digital assistant 125 provides a message 320 in the interaction interface to request the user to provide a examination result of “blood routine”. The user 140 uploads the PDF file 322. By parsing the PDF file 322, for example, by extracting text contained in the PDF file 322 or based on the file name, it can be determined that the PDF file 322 does not relate to “blood routine”, so it can be determined that the file does not meet the health consultation requirement. The digital assistant 125 may provide prompt information 324 to indicate that the current file does not meet the requirement and prompt the user 140 to input a correct file.

In some embodiments, in an input interface (for example, an upload interface) of the target file, whether the target file meets the health consultation requirement can be automatically identified, and when the file that does not meet the health consultation requirement is identified, prompt information is provided in an input interface, so that the user can change the file to be uploaded in time. As shown in FIG. 3C, an input interface 302 may be provided for a user to select one or more files to upload. If the user selects files 340, 342, and 344 and clicks on the “Upload report” control 346, it may be identified whether the file to be uploaded by the user meets the medical consultation requirement. If it is detected that one or more files to be uploaded do not meet the medical consultation requirement, as shown in FIG. 3C, prompt information 348 may be provided in the input interface 302 to prompt that some or all of the files do not meet the medical consultation requirement.

In some embodiments, the files that do not meet the requirement may also be specifically labeled through the prompt information, which is particularly useful when the user selects a plurality of files to be uploaded at the same time, thereby facilitating the user to quickly screen and delete the files that do not meet the requirement. As shown in FIG. 3C, in response to determining that the files 342 and 344 do not meet the medical consultation requirement, prompt information 350 and 352 may be provided at corresponding locations of the file to label that the two files do not meet the requirement.

In some embodiments, when the user uploads or shoots a file in real time, prompt information may also be provided to guide the user to upload a correct health related file, which may reduce misconception and repeated attempts due to uploading a wrong file. As shown in FIG. 3D, when the user shoots a paper file, text prompt information 330 may be provided in the shooting interface 303 to prompt a requirement for an input file. Alternatively or additionally, a shooting prompt box 332 may also be provided to guide the user to shoot in a correct shooting angle and size, which helps the user to improve the uploading accuracy. Alternatively or additionally, an example viewing control 334 for shooting template may also be provided for the user to view an example file that meets the requirement.

In some embodiments, as previously described, the digital assistant 125 determines a reply based on the model. Some traditional medical report analysis tools have great limitations, mainly in that when analyzing examination reports, key numerical information is identified by a rule or in a template matching manner, and a general analysis result is given. However, these analysis tools cannot give detailed explanation for the meaning of the numerical value, the reason behind the numerical value, and possible consequences thereof, and cannot dynamically solve further problems from the user. In the embodiments of the present disclosure, by means of the digital assistant 125 and the model used therein, it is possible to effectively combine the file and the context of the text message, guide the user to upload correct pictures/files, and provide detailed medical report analysis, thereby improving the service quality of the digital doctor.

In some embodiments, the model used by the digital assistant 125 includes a deep learning-based model capable of processing multi-modal data inputs including text input and non-text input. That is, the model not only can support pure text input, but also can parse and process files in the non-text message format uploaded by the user directly, such as various medical report images or electronic files.

In some embodiments, when the analysis result of the digital assistant 125 for the target file is determined based on the information extracted from the target file, the information may be extracted from the target file based on the parsing technique corresponding to the format of the target file, and the analysis result for the target file may be determined based on the extracted information and the interaction context between the user 140 and the digital assistant 125. In some embodiments, the extracted information includes text information included in the target file. Alternatively or additionally, the extracted information includes image information in the target file. For example, for a file involving medical image examination, part or all of the content of the file may involve medical images, which is also important for medical analysis.

Depending on the format of the target file, different parsing techniques may be utilized. For example, if the target file includes an image file or video file, text information in the image or video file may be extracted based on the optical character recognition (OCR) technique. In some embodiments, if the target file includes a non-editable text format such as a PDF file, the text therein may be extracted by using a corresponding text extraction technique. In some embodiments, if the target file includes image information related to medical health, the image information of the corresponding area may be directly intercepted.

In some embodiments, a prompt word input for the model may be generated based on information extracted from the target file and provided to the model to obtain an output of the model. The prompt word input may include text information and image information. An analysis result for the target file may then be determined based on the output of the model. In some embodiments, for the information extracted from the target file, the key information may be screened therefrom as the model input, or all the information may be provided as the model input.

In some embodiments, in addition to obtaining a file meeting the requirement to provide the analysis result, the terminal device 110 may further provide a parsed version corresponding to the target file in the interaction interface. The parsed version includes information extracted from the target file, such as text information and image information. For example, in the examples of FIG. 2C and FIG. 2D, for the image file 242 and the PDF file 246 related to the blood routine, the parsing results of the files are returned to the interaction interface as the message 248.

In some embodiments, the parsed version corresponding to the target file may include a structured representation of the information extracted from the target file. Organizing the information in the target file in a structured manner may facilitate viewing by the user. In some embodiments, the specific format of the information may be based on the information layout format in the target text, which is not limited herein.

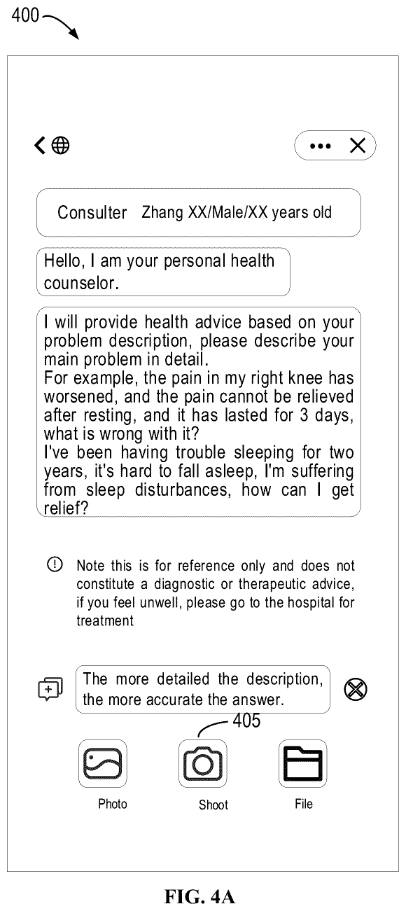

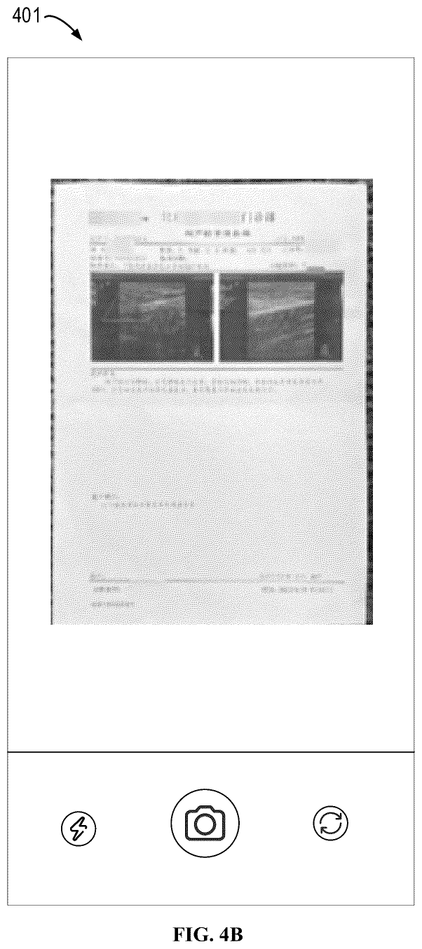

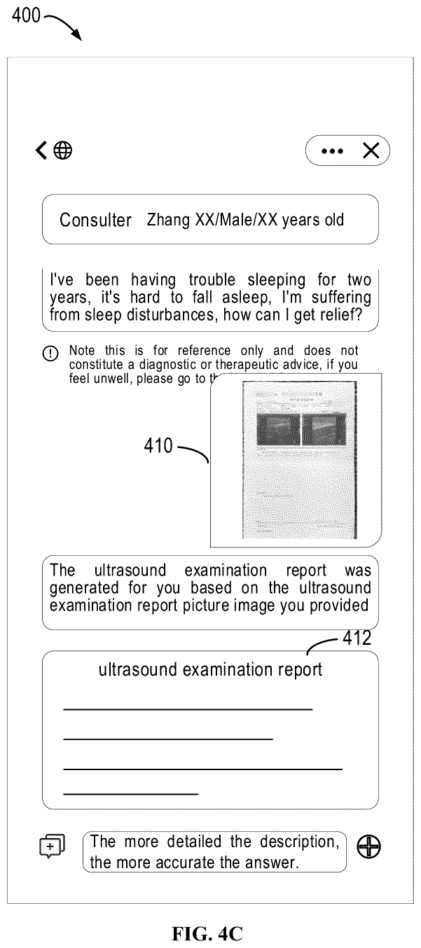

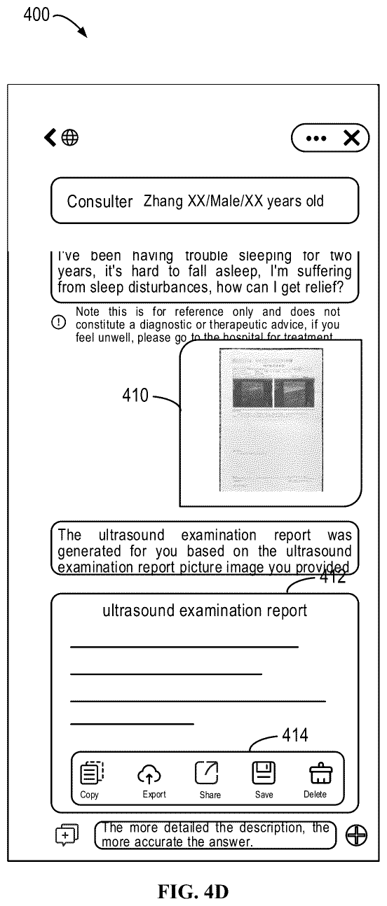

In an interaction interface 400 shown in FIG. 4A, the user triggers a “Shooting” control 405 to shot a file to be input. The image file 410 shot by the shooting interface 401 is uploaded to the interaction interface 401 shown in FIG. 4B. By parsing the image file 410, the formatted parsed version 412 of the file is provided in the interaction interface.

Since the information contained in the parsed version corresponding to the target file is an intermediate product in a process of generating the analysis result of the file, providing the parsed version to the user 140 may facilitate subsequent use of the target file by the user 140. In addition, by returning the analysis result back, the user 140 may also be guided to verify and modify the analysis result, so as to obtain a more accurate analysis result. For example, if the user 140 determines that parsing of some information in the returned analysis result is incorrect by viewing the same, the user 140 may further input an interaction message in the interaction interface to request to correct some information therein. In this way, the digital assistant 125 may determine the analysis result for the target file based on the corrected information and the interaction context.

In some embodiments, for the parsed version corresponding to the provided target file, the user may be supported for further interaction, including one or more of the following interacting operations on the parsed version: a copying operation, an exporting to local operation, a sharing operation, a printing operation, and a deleting operation, and the like. A specific interacting operation may be selected according to actual application requirements. As such, the user 140 can better utilize the target file in subsequent health consultation or otherwise.

Specifically, in some embodiments, in response to detecting an interaction triggering operation on the parsed version corresponding to the target file, the terminal device 110 may provide at least one interaction option for the parsed version, each interaction option corresponding to an interacting operation for the parsed version. The interaction triggering operation may include an appropriate triggering operation such as long press, double click, click, and right click. In response to receiving a selection for a target interaction option in the at least one interaction option, the terminal device 110 may perform a target interacting operation corresponding to the target interaction option on the parsed version, such as a copying operation, an exporting to local operation, a sharing operation, a printing operation, and a deleting operation. For example, in response to detecting a long press operation in the interaction interface 400 on the parsed version 412 of FIG. 4C, as shown in FIG. 4D, an interaction option box 414 for the parsed version is presented, including a plurality of selectable interaction options, such as Delete, Export, Share, Printing, Delete, and the like. The user 140 may select a certain interacting operation as needed.

According to the embodiment of the present disclosure, in combination with the file parsing technique, the file provided in the interaction between the user and the digital assistant can be intelligently analyzed to determine whether the file relates to medical related information or related to the consultation context, so that the reply of the digital assistant will not be confused, and the analysis of the digital assistant is more accurate, professional and targeted. By means of the model supporting multi-modal input, various user inputs can be more conveniently processed, and an accurate reply is provided in combination with the context. In some embodiments, the user may also be guided to upload a file meeting the requirement, thereby improving interaction efficiency and user friendliness. In addition, in some embodiments, the text uploaded by the user may also be further structured as interactable information or structured information, which can be convenient for the user to use directly in subsequent health consultations.

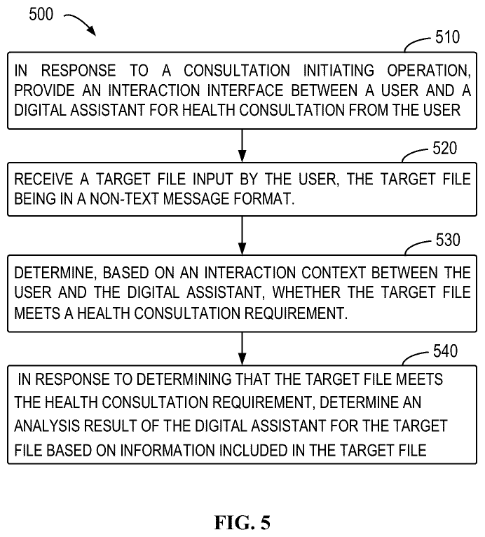

FIG. 5 illustrates a flowchart of a process 500 for information interaction according to some embodiments of the present disclosure. The process 500 may be implemented at the terminal device 110. The process 500 is described below with reference to FIG. 1.

At block 510, the terminal device 110 provides, in response to a consultation initiating operation, an interaction interface between a user and a digital assistant for health consultation from the user.

At block 520, the terminal device 110 receives a target file input by the user, the target file being in a non-text message format.

At block 530, the terminal device 110 determines, based on an interaction context between the user and the digital assistant, whether the target file meets a health consultation requirement.

At block 540, in response to determining that the target file meets the health consultation requirement, the terminal device 110 determines an analysis result of the digital assistant for the target file based on information included in the target file.

In some embodiments, determining whether the target file meets the health consultation requirement includes: determining that the target file meets the health consultation requirement by determining at least one of the following: determining that the target file includes medical related information, determining that an information type included in the target file matches an information type requested from the user in the interaction context, and determining that an appearance layout of the target file meets a predetermined layout requirement.

In some embodiments, the process 500 further includes: providing prompt information to prompt the user to input a correct file in response to determining that the target file does not meet the health consultation requirement.

In some embodiments, providing the prompt information includes: providing the prompt information as an interaction message in the interaction interface. In some embodiments, providing the prompt information includes: providing the prompt information in an input interface of the target file.

In some embodiments, determining the analysis result of the digital assistant for the target file based on information extracted from the target file includes: extracting the information from the target file based on a parsing technique corresponding to a format of the target file; and determining the analysis result for the target file based on the extracted information and the interaction context between the user and the digital assistant.

In some embodiments, the process 500 further includes: providing a parsed version corresponding to the target file in the interaction interface, the parsed version including the information extracted from the target file.

In some embodiments, the parsed version corresponding to the target file includes a structured representation of the information extracted from the target file.

In some embodiments, the process 500 further includes: in response to detecting an interaction triggering operation on the parsed version corresponding to the target file, providing at least one interaction option for the parsed version, each interaction option corresponding to an interacting operation for the parsed version; and in response to receiving a selection for a target interaction option in the at least one interaction option, performing a target interacting operation corresponding to the target interaction option on the parsed version.

In some embodiments, at least one interacting operation corresponding to the at least one interaction option includes at least one of: a copying operation, an exporting to local operation, a sharing operation, a printing operation, and a deleting operation.

In some embodiments, the target file includes a file uploaded or captured in real time by the user.

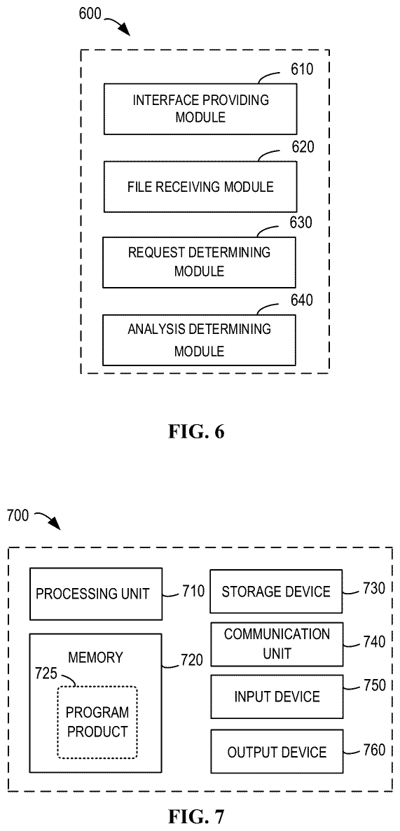

FIG. 6 is a schematic structural block diagram of an apparatus 600 for information interaction according to some embodiments of the present disclosure. The apparatus 600 may be implemented or included in the terminal device 110. Various modules/components in the apparatus 600 may be implemented by hardware, software, firmware, or any combination thereof.

As shown in the drawings, the apparatus 600 includes an interface providing module 610 configured to provide, in response to a consultation initiating operation, an interaction interface between a user and a digital assistant for health consultation from the user. The apparatus 600 further includes a file receiving module 620 configured to receive a target file input by the user, the target file being in a non-text message format. The apparatus 600 further includes a request determining module 630 configured to determine, based on an interaction context between the user and the digital assistant, whether the target file meets a health consultation requirement. The apparatus 600 further includes an analysis determining module 640 configured to determine, in response to determining that the target file meets the health consultation requirement, an analysis result of the digital assistant for the target file based on information included in the target file.

In some embodiments, the request determining module 630 is configured to determine that the target file meets the health consultation requirement by determining at least one of the following: determining that the target file includes medical related information, determining that an information type included in the target file matches an information type requested from the user in the interaction context, and determining that an appearance layout of the target file meets a predetermined layout requirement.

In some embodiments, the apparatus 600 further includes a prompting module configured to provide, in response to determining that the target file does not meet the health consultation requirement, prompt information to prompt the user to input a correct file.

In some embodiments, the prompting module is configured to provide the prompt information as an interaction message in the interaction interface. In some embodiments, the prompting module is configured to provide the prompt information in an input interface of the target file.

In some embodiments, the analysis determining module 640 includes: an information extracting module configured to extract the information from the target file based on a parsing technique corresponding to a format of the target file; and an information based analyzing and determining module configured to determine the analysis result for the target file based on the extracted information and the interaction context between the user and the digital assistant.

In some embodiments, the apparatus 600 further includes a parsing providing module configured to provide a parsed version corresponding to the target file in the interaction interface, the parsed version including the information extracted from the target file.

In some embodiments, the parsed version corresponding to the target file includes a structured representation of the information extracted from the target file.

In some embodiments, the apparatus 600 further includes: an interaction option providing module configured to provide, in response to detecting an interaction triggering operation on the parsed version corresponding to the target file, at least one interaction option for the parsed version, each interaction option corresponding to an interacting operation for the parsed version; and an interaction performing module configured to perform, in response to receiving a selection for a target interaction option in the at least one interaction option, a target interacting operation corresponding to the target interaction option on the parsed version.

In some embodiments, at least one interacting operation corresponding to the at least one interaction option comprises at least one of: a copying operation, an exporting to local operation, a sharing operation, a printing operation, and a deleting operation.

In some embodiments, the target file includes a file uploaded or captured in real time by the user.

FIG. 7 illustrates a block diagram of an electronic device 700 in which one or more embodiments of the present disclosure may be implemented. It shall be understood that the electronic device 700 shown in FIG. 7 is merely exemplary and should not constitute any limitation on the functionality and scope of the embodiments described herein. The electronic device 700 shown in FIG. 7 may be used to implement the terminal device 110 or the apparatus 600 of FIG. 6.

As shown in FIG. 7, the electronic device 700 is in the form of a general-purpose computing device. Components of the electronic device 700 may include, but are not limited to, one or more processors or processing units 710, a memory 720, a storage device 730, one or more communications units 740, one or more input devices 750, and one or more output devices 760. The processing unit 710 may be an actual or virtual processor and can perform various processes according to programs stored in the memory 720. In a multiprocessor system, a plurality of processing units execute computer executable instructions in parallel, so as to improve the parallel processing capability of the electronic device 700.

The electronic device 700 typically includes a number of computer storage media. Such media may be any available media that are accessible by electronic device 700, including, but not limited to, volatile and non-volatile media, removable and non-removable media. The memory 720 may be a volatile memory (e. g., a register, cache, random access memory (RAM)), non-volatile memory (e.g., read-only memory (ROM), electrically erasable programmable read-only memory (EEPROM), flash memory), or some combination thereof. The storage device 730 may be a removable or non-removable medium and may include a machine-readable medium such as a flash drive, a magnetic disk, or any other medium that can be used to store information and/or data and that can be accessed within the electronic device 700.

The electronic device 700 may further include additional removable/non-removable, volatile/nonvolatile storage media. Although not shown in FIG. 7, a magnetic disk drive for reading from or writing to a removable, nonvolatile magnetic disk such as a “floppy disk” and an optical disk drive for reading from or writing to a removable, nonvolatile optical disk may be provided. In these cases, each drive may be connected to a bus (not shown) by one or more data media interfaces. The memory 720 may include a computer program product 725 having one or more program modules configured to perform various methods or actions of various embodiments of the present disclosure.

The communication unit 740 implements communication with other electronic devices through a communication medium. In addition, functions of components of the electronic device 1000 may be implemented by a single computing cluster or a plurality of computing machines, and these computing machines can communicate through a communication connection. Thus, the electronic device 700 may operate in a networked environment using logical connections to one or more other servers, network personal computers (PCs), or another network node.

The input device 750 may be one or more input devices such as a mouse, keyboard, trackball, etc. The output device 760 may be one or more output devices such as a display, speaker, printer, etc. The electronic device 700 may also communicate with one or more external devices (not shown) such as a storage device, a display device, or the like through the communication unit 740 as required, and communicate with one or more devices that enable a user to interact with the electronic device 700, or communicate with any device (e. g., a network card, a modem, or the like) that enables the electronic device 700 to communicate with one or more other electronic devices. Such communication may be performed via an input/output (I/O) interface (not shown).

According to an exemplary embodiment of the present disclosure, a computer readable storage medium is provided, on which a computer-executable instruction is stored, wherein the computer executable instruction is executed by a processor to implement the above-described method. According to an exemplary embodiment of the present disclosure, there is also provided a computer program product, which is tangibly stored on a non-transitory computer readable medium and includes computer-executable instructions that are executed by a processor to implement the method described above.

Aspects of the present disclosure are described herein with reference to flowchart and/or block diagrams of methods, apparatus, devices and computer program products implemented in accordance with the present disclosure. It will be understood that each block of the flowcharts and/or block diagrams and combinations of blocks in the flowchart and/or block diagrams can be implemented by computer readable program instructions.

These computer readable program instructions may be provided to a processing unit of a general-purpose computer, special purpose computer, or other programmable data processing apparatus to produce a machine, such that the instructions, which execute via the processing unit of the computer or other programmable data processing apparatus, create means for implementing the functions/actions specified in one or more blocks of the flowchart and/or block diagrams. These computer readable program instructions may also be stored in a computer readable storage medium that can direct a computer, a programmable data processing apparatus, and/or other devices to function in a particular manner, such that the computer readable medium storing the instructions includes an article of manufacture including instructions which implement various aspects of the functions/actions specified in one or more blocks of the flowchart and/or block diagrams.

The computer readable program instructions may be loaded onto a computer, other programmable data processing apparatus, or other devices, causing a series of operational steps to be performed on a computer, other programmable data processing apparatus, or other devices, to produce a computer implemented process such that the instructions, when being executed on the computer, other programmable data processing apparatus, or other devices, implement the functions/actions specified in one or more blocks of the flowchart and/or block diagrams.

The flowcharts and block diagrams in the drawings illustrate the architecture, functionality, and operations of possible implementations of the systems, methods and computer program products according to various implementations of the present disclosure. In this regard, each block in the flowchart or block diagram may represent a module, segment, or portion of instructions which includes one or more executable instructions for implementing the specified logical function(s). In some alternative implementations, the functions marked in the blocks may occur in a different order than those marked in the drawings. For example, two consecutive blocks may actually be executed in parallel, or they may sometimes be executed in reverse order, depending on the function involved. It should also be noted that each block in the block diagrams and/or flowcharts, as well as combinations of blocks in the block diagrams and/or flowcharts, may be implemented using a dedicated hardware-based system that performs the specified function or operations, or may be implemented using a combination of dedicated hardware and computer instructions.

Various implementations of the disclosure have been described as above, the foregoing description is exemplary, not exhaustive, and the present application is not limited to the implementations as disclosed. Many modifications and variations will be apparent to those of ordinary skill in the art without departing from the scope and spirit of the implementations as described. The selection of terms used herein is intended to best explain the principles of the implementations, the practical application, or improvements to technologies in the marketplace, or to enable those skilled in the art to understand various embodiments disclosed herein.

I/We claim:

1. A method for information interaction, comprising:
in response to a consultation initiating operation, providing an interaction interface between a user and a digital assistant for health consultation from the user;
receiving a target file input by the user, the target file being in a non-text message format;
determining, based on an interaction context between the user and the digital assistant, whether the target file meets a health consultation requirement; and
in response to determining that the target file meets the health consultation requirement, determining an analysis result of the digital assistant for the target file based on information included in the target file.

2. The method of claim 1, wherein determining whether the target file meets the health consultation requirement comprises:
determining that the target file meets the health consultation requirement by determining at least one of the following:
determining that the target file includes medical related information;
determining that an information type included in the target file matches an information type requested from the user in the interaction context; and
determining that an appearance layout of the target file meets a predetermined layout requirement.

3. The method of claim 1, further comprising:
in response to determining that the target file does not meet the health consultation requirement, providing prompt information to prompt the user to input a correct file.

4. The method of claim 1, wherein determining the analysis result of the digital assistant for the target file based on information extracted from the target file comprises:
extracting the information from the target file based on a parsing technique corresponding to a format of the target file; and
determining the analysis result for the target file based on the extracted information and the interaction context between the user and the digital assistant.

5. The method of claim 1, further comprising:
providing a parsed version corresponding to the target file in the interaction interface, the parsed version comprising the information extracted from the target file.

6. The method of claim 5, wherein the parsed version corresponding to the target file comprises a structured representation of the information extracted from the target file.

7. The method of claim 5, further comprising:
in response to detecting an interaction triggering operation on the parsed version corresponding to the target file, providing at least one interaction option for the parsed version, each interaction option corresponding to an interacting operation for the parsed version; and
in response to receiving a selection for a target interaction option in the at least one interaction option, performing a target interacting operation corresponding to the target interaction option on the parsed version.

8. The method of claim 7, wherein at least one interacting operation corresponding to the at least one interaction option comprises at least one of: a copying operation, an exporting to local operation, a sharing operation, a printing operation, and a deleting operation.

9. The method of claim 1, wherein the target file comprises a file uploaded or captured in real time by the user.

10. An electronic device, comprising:
at least one processing unit; and
at least one memory coupled to the at least one processing unit and storing instructions for execution by the at least one processing unit, the instructions, when executed by the at least one processing unit, causing the device to perform operations comprising:
in response to a consultation initiating operation, providing an interaction interface between a user and a digital assistant for health consultation from the user;
receiving a target file input by the user, the target file being in a non-text message format;
determining, based on an interaction context between the user and the digital assistant, whether the target file meets a health consultation requirement; and
in response to determining that the target file meets the health consultation requirement, determining an analysis result of the digital assistant for the target file based on information included in the target file.

11. The electronic device of claim 10, wherein determining whether the target file meets the health consultation requirement comprises:
determining that the target file meets the health consultation requirement by determining at least one of the following:
determining that the target file includes medical related information;
determining that an information type included in the target file matches an information type requested from the user in the interaction context; and
determining that an appearance layout of the target file meets a predetermined layout requirement.

12. The electronic device of claim 10, wherein the operations further comprise:
in response to determining that the target file does not meet the health consultation requirement, providing prompt information to prompt the user to input a correct file.

13. The electronic device of claim 10, wherein determining the analysis result of the digital assistant for the target file based on information extracted from the target file comprises:
extracting the information from the target file based on a parsing technique corresponding to a format of the target file; and
determining the analysis result for the target file based on the extracted information and the interaction context between the user and the digital assistant.

14. The electronic device of claim 10, wherein the operations further comprise:
providing a parsed version corresponding to the target file in the interaction interface, the parsed version comprising the information extracted from the target file.

15. The electronic device of claim 14, wherein the parsed version corresponding to the target file comprises a structured representation of the information extracted from the target file.

16. The electronic device of claim 14, wherein the operations further comprise:
in response to detecting an interaction triggering operation on the parsed version corresponding to the target file, providing at least one interaction option for the parsed version, each interaction option corresponding to an interacting operation for the parsed version; and
in response to receiving a selection for a target interaction option in the at least one interaction option, performing a target interacting operation corresponding to the target interaction option on the parsed version.

17. The electronic device of claim 16, wherein at least one interacting operation corresponding to the at least one interaction option comprises at least one of: a copying operation, an exporting to local operation, a sharing operation, a printing operation, and a deleting operation.

18. The electronic device of claim 10, wherein the target file comprises a file uploaded or captured in real time by the user.

19. A non-transitory computer readable storage medium having a computer program stored thereon which, when executed by a processor, perform operations comprising:
in response to a consultation initiating operation, providing an interaction interface between a user and a digital assistant for health consultation from the user;
receiving a target file input by the user, the target file being in a non-text message format;
determining, based on an interaction context between the user and the digital assistant, whether the target file meets a health consultation requirement; and
in response to determining that the target file meets the health consultation requirement, determining an analysis result of the digital assistant for the target file based on information included in the target file.

20. The non-transitory computer readable storage medium of claim 19, wherein determining whether the target file meets the health consultation requirement comprises:
determining that the target file meets the health consultation requirement by determining at least one of the following:
determining that the target file includes medical related information;
determining that an information type included in the target file matches an information type requested from the user in the interaction context; and
determining that an appearance layout of the target file meets a predetermined layout requirement.
